(19) United States
(12) Patent Application Publication
(10) Pub. No.: US 2008/0082077 A1
(43) Pub. Date: Apr. 3, 2008

(54) SYSTEM AND METHOD FOR FLOW RATE CONTROL

(76) Inventor: David Lloyd Williams, Newport Beach, CA (US)

Correspondence Address: ALCON, IP LEGAL, TB4-8, 6201 SOUTH FREEWAY, FORT WORTH, TX, 76134, US

(21) Appl. No.: 11/860,609

(22) Filed: Sep. 25, 2007

Related U.S. Application Data
(60) Provisional application No. 60/848,473, filed on Sep. 29, 2006.

(51) Int. Cl. A61M 31/00

(52) U.S. Cl. 604/506; 604/131

(57) ABSTRACT

Embodiments of the present invention provide an apparatus and method controlling the flow of fluid in a surgical cassette. One embodiment of the present invention includes surgical system comprising a surgical console and a surgical cassette. The surgical console and surgical cassette can each contain portions of a valve system that can be controlled to provide proportional flow control of fluid to a chamber in the cassette.

### CROSS-REFERENCE TO RELATED APPLICATIONS

This application claims priority under 35 U.S.C. §119 to U.S. Provisional Patent Application No. 60/848,473, filed Sep. 29, 2006, the entire contents of which are incorporated herein by reference.

### TECHNICAL FIELD OF THE INVENTION

The present invention relates to surgical systems and methods. More particularly, the present invention relates to systems and methods for controlling fluid flow. Even more particularly, embodiments of the present invention relate to systems and methods for a proportional flow valve in a surgical system.

### BACKGROUND OF THE INVENTION

The human eye can suffer a number of maladies causing mild deterioration to complete loss of vision. While contact lenses and eyeglasses can compensate for some ailments, ophthalmic surgery is required for others. Generally, ophthalmic surgery is classified into posterior segment procedures, such as vitreoretinal surgery, anterior segment procedures, such as cataract surgery, and combined anterior and posterior segment procedures.

The surgical instrumentation used for ophthalmic surgery can be specialized for posterior segment procedures or anterior segment procedures or support both. In any case, the surgical instrumentation often requires the use of associated consumables such as surgical cassettes, fluid bottles/bags, tubing, hand piece tips and other consumables.

A surgical cassette can provide a variety of functions depending on the procedure and surgical instrumentation. For example, surgical cassettes for vitreoretinal surgical procedures help manage irrigation and aspiration flows into and out of a surgical site. The cassette acts as the interface between surgical instrumentation and the patient. It delivers pressurized infusion and aspiration flows into and out of the eye.

The flow of fluid to the infusion chamber of a surgical set is typically controlled by a simple on/off valve. The sharp closing of a valve, however, can cause turbulence or shock in the fluid thereby causing undesirable pressure surges into the eye, incorrect measurement of the fluid level in the infusion chamber, or other deleterious effects.

### SUMMARY OF THE INVENTION

Embodiments of the present invention provide an apparatus and method for controlling the flow of fluid in a surgical cassette. One embodiment of the present invention includes a surgical system comprising a surgical console and a surgical cassette. The surgical cassette, according to one embodiment, can comprise a valve, comprising a valve seat and a valve seal movable between a fully opened position and fully closed position. The valve seat and valve seal define a valve chamber. The surgical cassette can also comprise a first flow passage leading to the valve chamber and a second flow passage leading to a fluid chamber of the surgical cassette. The surgical console can comprise a cassette receiver to receive the surgical cassette, a sensor system, an actuator positioned to assert a force on the valve seal when the surgical cassette is inserted in the cassette receiver and a controller coupled to the sensor system and the actuator. The controller can be configured to receive an input from the sensor system and generate a control signal to the actuator to increase or decrease the force asserted on the valve seal to control a flow rate of fluid to the fluid chamber according to a specified flow rate.

Another embodiment of the present invention includes a method comprising determining a flow rate of a fluid in a surgical cassette inserted in a surgical console, comparing the measured flow rate to a setpoint flow rate, signaling an actuator of the surgical console to assert more or less force on a valve seal of the surgical cassette based on the difference between the measured flow rate and the setpoint flow rate and moving the valve seal of the surgical cassette to increase or decrease the flow rate.

Yet another embodiment of the present invention can comprise a computer program product comprising a set of computer instructions stored on a computer readable medium. The set of computer instructions can comprise instructions executable by a processor to determine a flow rate of fluid in a surgical cassette, compare the flow rate to a setpoint, if the flow rate is greater than the setpoint, generate a control signal to cause an actuator to assert more force on a valve seal of the surgical cassette to decrease the flow rate and if the flow rate is less than the setpoint, generate the control signal to cause the actuator to assert less force on the valve seal of the surgical cassette to increase the flow rate.

In the embodiments of the present invention, the valve can comprise a valve configured such that increasing the asserted force on the valve seal will increase the flow rate and decreasing the asserted force on the valve seat will decrease the flow rate.

Embodiments of the present invention provide an advantage over prior art systems and methods of flow control in surgical cassettes by allowing flow rate to be controlled independent of controlling pressure of a source fluid. Embodiments of the present invention can comprise a tapered valve seat and/or a tapered valve seal. Further, embodiments of the present invention can be implemented as normally-closed valves, as will be familiar to those having skill in the art. In some embodiments, the surgical system can comprise tapered valve actuators to control (increase/decrease) flow rate through valves that have non-tapered seats and/or seals.

Embodiments of the present invention provide another advantage over prior art systems and methods of flow control, by allowing for precise flow control and gentle closing of fluid valves to reduce turbulence and pressure spikes.

### BRIEF DESCRIPTION OF THE FIGURES

A more complete understanding of the present invention and the advantages thereof may be acquired by referring to the following description, taken in conjunction with the accompanying drawings in which like reference numbers indicate like features and wherein:

FIG. 1 is a diagrammatic representation of one embodiment of a surgical console;

FIG. 2 is a diagrammatic representation of one embodiment of a surgical cassette;

FIGS. 3A-3C are diagrammatic representations of various embodiments of valve seats;

FIG. 4 is a diagrammatic representation of one embodiment of a cassette receiver;

FIG. 5 is a diagrammatic representation of one embodiment of a proportional valve system for a surgical system;

FIG. 6 is a diagrammatic representation of one embodiment of a method for flow control.

### DETAILED DESCRIPTION

Preferred embodiments of the invention are illustrated in the FIGURES, like numerals being used to refer to like and corresponding parts of the various drawings.

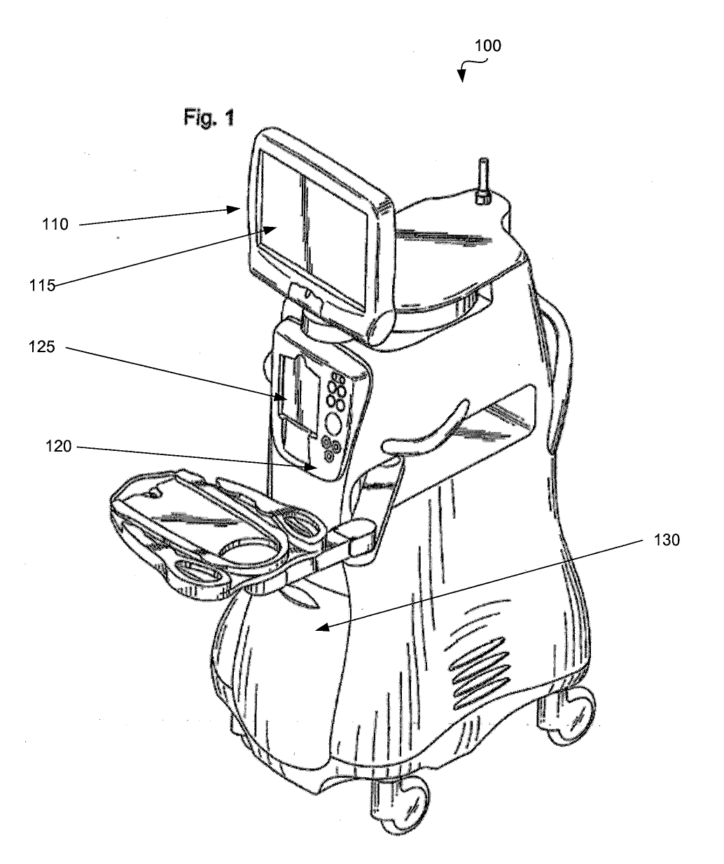

FIG. 1 is a diagrammatic representation of one embodiment of an ophthalmic surgical console 100. Surgical console 100 can include a swivel monitor 110 that has touch screen 115. Swivel monitor 110 can be positioned in a variety of orientations for whomever needs to see touch screen 115. Swivel monitor 110 can swing from side to side, as well as rotate and tilt. Touch screen 115 provides a graphical user interface (“GUI”) that allows a user to interact with console 100.

Surgical console 100 also includes a connection panel 120 used to connect various tools and consumables to surgical console 100. Connection panel 120 can include, for example, a coagulation connector, connectors for various hand pieces, and a cassette receiver 125. Surgical console 100 can also include a variety of user friendly features, such as a foot pedal control (e.g., stored behind panel 130) and other features.

In operation, a cassette (not shown) can be placed in cassette receiver 125. A clamp in surgical console 100 clamps the cassette in place to minimize movement of the cassette during use. The clamp can clamp the top and bottom of the cassette, the sides of the cassette or otherwise clamp the cassette.

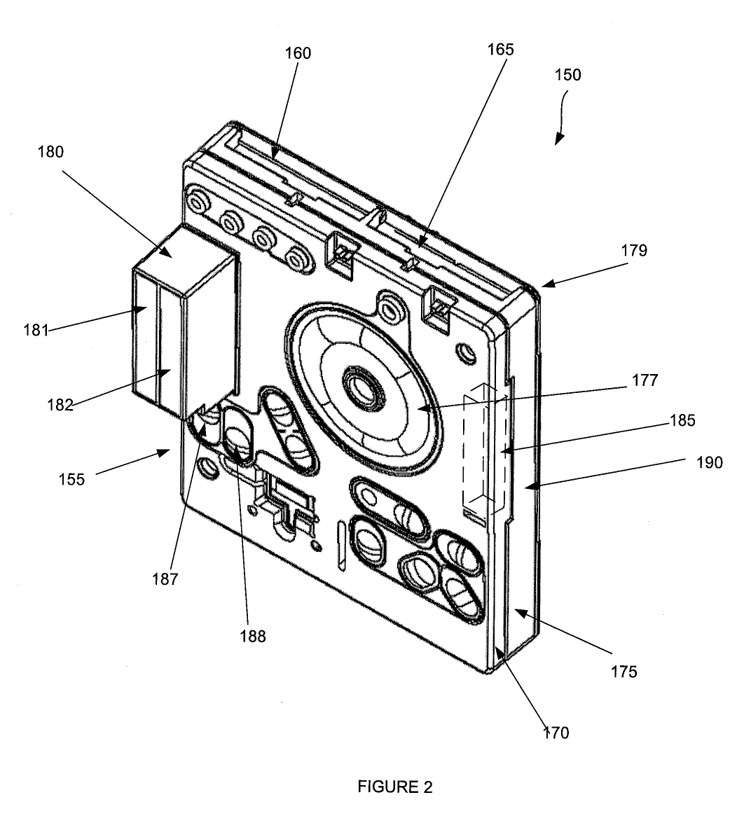

FIG. 2 is a diagrammatic representation of one embodiment of a surgical cassette 150. Cassette 150 can provide a closed system fluidic device that can be discarded following a surgical procedure. Cassette 150 can include a cassette body 155 and portions that interface with the clamp (e.g., indicated generally at clamping zones 160 and 165) projecting from the cassette body 155. Cassette 150 can be formed of ABS plastic or other suitable material. In the embodiment shown, cassette 150 is formed from three primary sections: an inner or surgical console interface section 170 that faces the surgical console when cassette 150 is inserted into surgical console 100, a middle section 175 and a cover plate 179. The various sections of cassette 150 can be coupled together via a press fit, interlocking tabs, chemical bonding, thermal bonding, mechanical fasteners or other attachment mechanism known in the art. In other embodiments, cassette 150 can be formed of a single piece or multiple pieces.

Surgical console interface section 170 can face the console during use and provide an interface for fluid flow channels (e.g., flow channel 177 for the peristaltic pump provided by an elastomeric pump membrane), valves (e.g., infusion/aspiration valves), and other features to manage fluid flow. Cassette 150 can also attach to a fluid bag (not shown) to collect fluids during a procedure.

Surgical cassette 150, according to various embodiments of the present invention, includes chambers to hold fluids for aspiration and infusion. For example, chamber cartridge 180 can include two infusion chambers 181/182. A third chamber 185 can be internal to cassette 150 on the opposite side of cassette 150 from chamber cartridge 180 (e.g., at the side of cassette 150 indicated by 190). According to one embodiment, flow of fluid to infusion chambers 181/182 can be controlled by proportional valves. The proportional valves can include features of surgical cassette 150 and features of the corresponding surgical console. With respect to surgical cassette 150, the proportional valve for controlling flow to infusion chamber 181 can include valve seal 187 and the proportional valve for controlling flow to infusion chamber 182 can include valve seal 188. As described below, each valve seal 187/188 can be moved toward a corresponding valve seat by a corresponding actuator in surgical system 100 to fully or partially close a valve inlet or outlet. Valve seals 187/188 can be formed of separate pieces or a single piece of an elastomeric material and can return to approximately their original shapes when the forces applied by the actuators are removed.

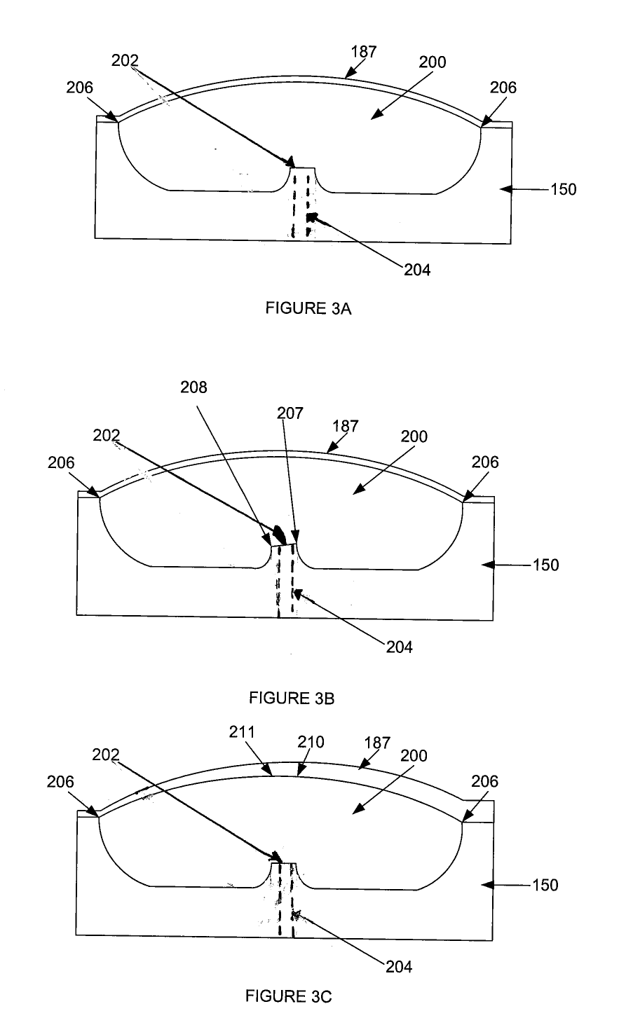

The valve seats of the valves can have various configurations. FIGS. 3A-3C are diagrammatic representations of top views of example configurations for the cassette portion of a proportional valve. In each of the examples of FIGS. 3A-3C, the valve includes a valve chamber 200 defined by the valve seal (e.g., valve seal 187) and a valve seat 202 disposed in cassette 150. An inlet flow passage 204 leads fluid into the valve chamber 200 and an outlet flow passage (not shown) leads out of valve chamber 200 (e.g. from the top, sides or bottom of valve chamber 200) to an infusion chamber (e.g., infusion chamber 181). Preferably, but not necessarily, valve seal 187 is coupled to the body of cassette 150 in a manner that does not allow fluid to leak out of valve chamber 200 at edge 206. Valve seal 187 can be coupled to the body of cassette 150 according to any suitable mechanism including joining through mechanical or chemical bonding. According to other embodiments, valve seal 187 is coupled to cassette 150 by sandwiching a sheet of material that includes valve seal 187 between two portions of cassette 150 that are joined together (e.g., by press fitting or other joining mechanism).

In FIG. 3A, valve seat 202 is shaped to be flat where flow passage 204 intersects valve chamber 200. In operation, valve seal 187 is moved towards valve seat 202 by an actuator, thereby decreasing the distance between the entrance to flow passage 204 and valve seal 187. As this distance becomes smaller, the flow rate through valve chamber 200 will decrease until flow ultimately stops when valve seal 187 contacts valve seat 202 with sufficient force to seal the entrance to flow passage 204. By controlling the position of valve seal 187 between a fully opened position (i.e., the position configured to allow the most flow when the cassette is in use) and a fully closed position, the flow rate of fluid can be controlled.

In FIG. 3B, valve seat 202 is tapered at the entrance of flow passage 204 so that one edge of the entrance is closed before the other edge. In FIG. 3B, for example, point 207 is closer to valve seal 187 than point 208 when valve seal 187 is in its fully opened position. As valve seal 187 moves toward valve seat 202, valve seal 187 will contact point 207 first, but will have to move further to contact point 208 to fully seal the entrance of flow passage 204. This causes the usable opening of flow passage 204 to vary depending on how much force is applied to valve seal 187, thereby allowing for finer control of flow rate near the end of valve seal 187's range of motion.

In FIG. 3C, valve seal 187 is tapered so that it will contact valve seat 202 on one edge of the entrance of flow passage 204 before contacting valve seat 202 on the other edge. In the example of FIG. 3C, valve seal 187 is thicker at point 210 than point 211 so that point 210 will contact valve seat 202 first. Again, this causes the usable opening of flow passage 204 to vary depending on how much force is applied to valve seal 187 once valve seal 187 begins to contact valve seat 202.

Although FIGS. 3A-3C describe specific embodiments of valve configuration, these are provided by way of example and other configurations can be used. For example, both valve seat 202 and valve seal 187 can be tapered. Additionally, valve seal 187 can seal the outlet, rather than inlet flow passage. Furthermore, different valves in cassette 150 can have the same or different configurations.

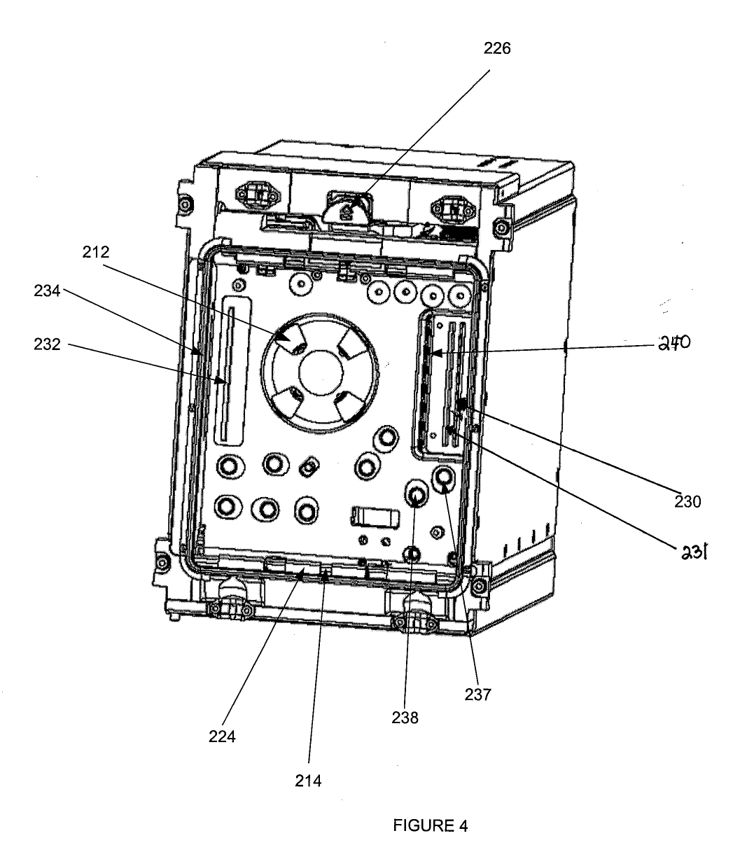

FIG. 4 is a diagrammatic representation of one embodiment of cassette receiver 125 without a cassette. Cassette receiver 125 can have various pneumatic input and output ports to interface with the surgical cassette. Cassette receiver 125 can further include an opening to allow peristaltic pump rollers 212 to contact the surgical cassette during operation. One embodiment of a peristaltic pump and complimentary cassette is described in U.S. patent application Ser. No. 6,293,926 to Sorensen, which is hereby fully incorporated by reference herein.

The surgical cassette is held in place by a clamp having a bottom rail 214 and a top rail (not shown). Each rail can have outer clamping fingers (e.g., clamp finger 224) that contact the cassette in corresponding clamping zones and inner clamping fingers to locate the cassette during insertion and push the cassette out of cassette receiver during release. A release button 226 is pressed to initiate release of the cassette from the clamp. Cassette receiver 125, according to one embodiment, can include linear light sources to project light into the walls of the cassette chambers and sensor arrays to detect the light refracted through the chamber (or reflected from the chamber wall). Each linear light source can include a plurality of light sources vertically arranged (i.e., to project light along vertically spaced transmission paths) and positioned to project light into a wall of the cassette. For example, linear light source 230 can project light into chambers 181/182. Linear light source 230 can contain a first set of light sources aligned to project light into chamber 181 and a second set of light sources arranged at a 90 degree angle (or other angle) from the first set of light sources to project light into chamber 182. Similarly, linear light source 232 can project light into the walls of chamber 185. Respective linear sensor arrays can receive light refracted through the chamber or reflected at the chamber surface. In this example, sensor array (not shown) can receive light from light source 230 projected at chamber 181, a sensor array located in wall 234 can receive light from light source 232 projected at chamber 185 and a sensor array in wall 240 can receive light from light source 231. Each sensor array can include vertically arranged portions to receive light through the wall of the cassette chamber. The vertically arranged portions can be, for example, pixels, separate sensors or other mechanisms for sensing illumination. One example of a linear sensor array is the TAOS TSL208R linear sensor array by Texas Advanced Optoelectronic Systems of Plano, Tex., which has a resolution of 200 dots per inch (DPI).

As described in U.S. patent application Ser. No. 11/477,032, entitled “System and Method of Non-Invasive Continuous Level Sensing,” filed Jun. 28, 2006, which is hereby fully incorporated by reference herein, the level and hence volume of fluid in a chamber can be determined by projecting light into the wall of the cassette and evaluating the light pattern detected by the corresponding linear sensor array. By tracking the change in volume over time, the volumetric or mass flow rate of fluid into/out of the chamber can be determined.

As noted above, the flow rate of fluid into a chamber can be regulated by a proportional valve that can include features in the surgical console. For example, surgical console 100 can include an actuator to apply a force to valve seal 187, thereby regulating flow of fluid into chamber 181 and an actuator to apply a force to valve seal 188, thereby regulating flow of fluid into chamber 182. The actuators, according to one embodiment, can include shaft 237 to contact and press valve seal 187 and shaft 238 to contact and press valve seal 188. The actuators can be pneumatic actuators, electromechanical actuators (such as a solenoid driven actuator) or other actuator configured to impart a force to valve seals 187/188. Depending on the amount of force applied, the valve will allow more or less flow.

The configuration of FIG. 4 is provided by way of example. The form factor of cassette receiver 125, placement and number of input/output ports and other features of cassette receiver 125 can depend on the surgical console 100, surgical procedure being performed or other factors.

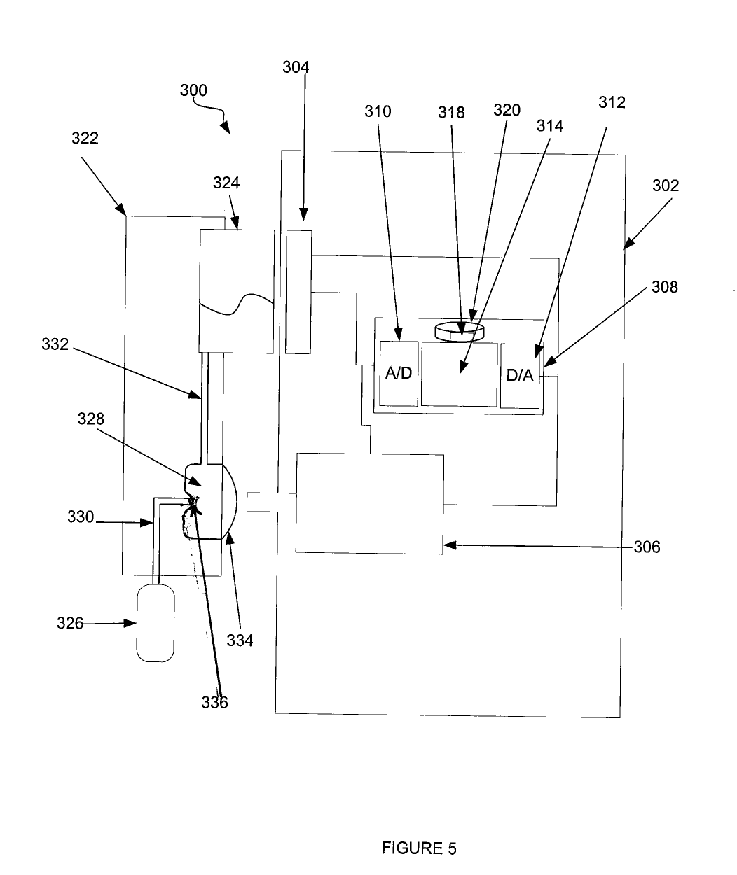

FIG. 5 is a diagrammatic representation of one embodiment of a proportional valve system for a surgical system 300 in which embodiments of proportional flow control according to the present invention can be implemented. According to the embodiment of FIG. 5, system 300 includes a surgical console 302 having a sensor system 304, an actuator 306 and a controller 308. Controller 308 includes can be any suitable controller known in the art including DSP, ASIC, RISK or CPU based controllers. Controller 308 can include an analog to digital (A/D) converter 310 to convert analog signals from sensor system 304 or actuator 306 to digital signals. Additionally, controller 308 can include a digital to analog (D/A) converter 312 to convert digital control signals to analog signals to control sensor system 304 or actuator 306. A processor 314, such as a DSP, ASIC, RISC, microcontroller or CPU or other suitable processor can access a set of instructions 318 on a computer readable memory 320. The computer readable memory 320 can be RAM, ROM, magnetic storage, optical storage or other suitable memory and can be onboard or be accessible by processor 314.

Surgical system 300 can further include surgical cassette 322 inserted into surgical console 302. Surgical cassette 322 can include a fluid chamber 324, such as an infusion chamber or other chamber that can act as a fluid reservoir for surgical instrumentation. Fluid from a fluid source 326 (e.g., a source bottle) is led to a valve chamber 328 via an inlet flow passage 330 and from valve chamber 328 to fluid chamber 324 via an outlet flow passage 332. Typically, fluid from fluid source 326 is under pressure to allow fluid to flow from fluid source 326 to fluid chamber 324. The flow rate of fluid flowing from fluid source 326 to fluid chamber 324 is controlled by movement of valve seal 334 towards valve seat 336. More particularly, as valve seal 334 moves towards valve seat 336, the flow rate will decrease for a given pressure applied to the fluid. The flow rate will continue to decrease as valve seal 334 partially closes the opening of flow passage 330 and will stop when valve seal 334 fully closes the opening of flow passage 330.

In operation, actuator 306 can apply force to valve seal 334 (e.g., through a shaft or other mechanism) to cause valve seal 334 to move towards valve seat 336 to seal the opening of inlet flow passage 330. The force to move seal 334 to a particular position between a fully opened position and a fully closed position can depend on the geometry and on the modulus of elasticity of valve seal 334 and other factors (e.g., the pressure of fluid pushing on valve seal 334). As fluid flows into valve chamber 328, sensor system 304, such as a non-invasive sensor system as described above, can detect the level of fluid in fluid chamber 324 and provide an indication of the level to controller 308 that can determine the flow rate of fluid into chamber 324. This can be done based, for example, on the change in level, volume, fluid mass or other change over time corresponding to the flow rate of fluid into chamber 324. Controller 308 can compare the flow rate of fluid to a setpoint and send control signals to actuator 306 to apply more or less force to valve seal 334 to increase or decrease the flow rate accordingly.

Controller 308 can implement various control schemes understood in the art, including, but not limited to, proportional flow control, proportional-derivative flow control, or proportional-integral-derivative flow control. That is, controller 308 can act as a P-controller, PD-controller, PID-controller or other controller known or developed in the art to generate signals to actuator 306 based on a comparison of a measured flow rate and a setpoint flow rate. At the occurrence of a particular event, such as the fluid level reaching a predefined level, controller 308 can signal actuator 306 to assert sufficient force on valve seal 334 to seal the opening to inlet flow passage 330.

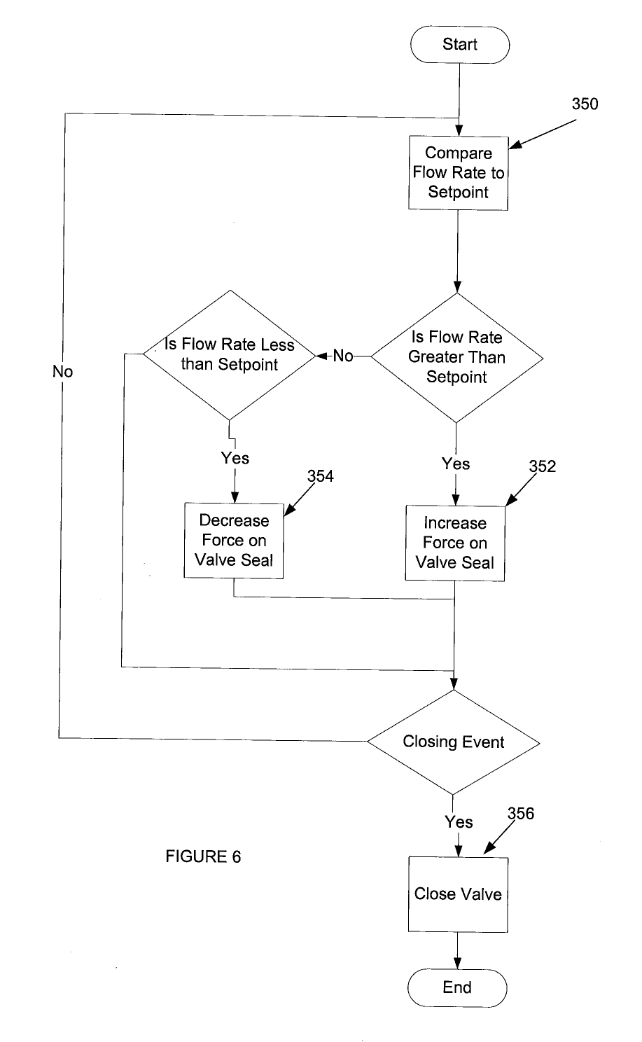

FIG. 6 is a flow chart illustrating one embodiment of a method for controlling flow to a chamber of a surgical cassette. The method of FIG. 6 can be facilitated through execution of computer instructions stored on a computer readable medium. At step 350, a controller can compare the flow rate of fluid into the chamber of a surgical cassette to a setpoint. If the flow rate is greater than a setpoint, the controller can signal an actuator to assert a greater force on a valve seal to decrease the flow rate (step 352). If, on the other hand, the flow rate of the fluid into the chamber is less than a setpoint, the controller can signal the actuator to assert less force on a valve seal to increase the flow rate (step 354). When a predetermined amount of fluid is in the chamber, the controller can signal the actuator to fully close the valve (step 356). It should be noted that, while in FIG. 6, the flow rate is adjusted if the flow does not match the setpoint, other control schemes can be implemented. For example, the flow rate can be adjusted based on whether the flow rate is outside of a set range about the setpoint. The steps of FIG. 6 can be repeated as needed or desired and the flow rate information updated continuously (e.g., at each processor cycle, instructions loop cycle or other period of time).

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed in the following claims.

What is claimed is:

1. A surgical system comprising:
a surgical cassette comprising:
a valve seat,
a valve seal movable between a fully opened position and fully closed position, wherein the valve seat and valve seal define a valve chamber and wherein the surgical cassette comprises a first flow passage leading to the valve chamber and a second flow passage leading to a fluid chamber of the surgical cassette;
a surgical console comprising:
a cassette receiver to receive the surgical cassette;
a sensor system;
an actuator positioned to assert a force on the valve seal when the surgical cassette is inserted in the cassette receiver; and
a controller coupled to the sensor system and the actuator, the controller configured to:
to receive an input from the sensor system;
generate a control signal to the actuator to increase or decrease the force asserted on the valve seal to control a flow rate of fluid to the fluid chamber according to a specified flow rate.

2. The surgical system of claim 1, wherein the input from the sensor system indicates a level of fluid in the fluid chamber.

3. The surgical system of claim 2, wherein the controller is further configured to:
determine the flow rate of the fluid based on a change in level of fluid over time in the fluid chamber;
compare the determined flow rate to a setpoint flow rate;
if the determined flow rate is greater than the setpoint flow rate, generate the control signal to the actuator to assert more force on the valve seal; and
if the determined flow rate is less than the setpoint flow rate, generate the control signal to actuator to cause the actuator to assert less force on the valve seal.

4. The surgical system of claim 1, wherein the controller is configured to cause the actuator to place the valve seal in a range of positions between the fully opened position and the fully closed position.

5. The surgical system of claim 1, wherein the valve seat is shaped so that an opening of the first flow passage is flat.

6. The surgical system of claim 1, wherein the valve seat is shaped so that the opening of the first flow passage is tapered.

7. The surgical system of claim 1, wherein the valve seal is tapered.

8. The surgical system of claim 1, wherein the first flow passage is an inlet flow passage to the fluid chamber.

9. The surgical system of claim 1, wherein the first flow passage is an outlet flow passage of the fluid chamber.

10. A method for controlling flow of fluid in a surgical cassette comprising:
determining a flow rate of fluid in the surgical cassette, wherein the surgical cassette is inserted in the surgical console;
comparing the measured flow rate to a setpoint flow rate;
signaling an actuator of the surgical console to assert more or less force on a valve seal of the surgical cassette based on the difference between the measured flow rate and the setpoint flow rate; and
moving the valve seal of the surgical cassette to increase or decrease the flow rate.

11. The method of claim 10, further comprising:
moving the valve seal to a position that partially closes the opening of a flow passage to a valve chamber through which fluid is flowing.

12. The method of claim 10, wherein determining the flow rate of the fluid comprises measuring the level of the fluid in a fluid chamber and determining the flow rate based on a change in the level over time.

13. The method of claim 12, wherein measuring the level of fluid in the fluid chamber comprises non-invasively measuring the level of fluid in the chamber.

14. The method of claim 10, further comprising stopping the flow of fluid at the occurrence of a predefined event.

15. The method of claim 14, wherein the predefined event is a level of fluid at the surgical cassette reaching a specified level.

16. A computer program product comprising a set of computer instructions stored on a computer readable medium, said set of computer instructions comprising instructions executable by a processor to:
determine a flow rate of fluid in a surgical cassette;
compare the flow rate to a setpoint;
if the flow rate is greater than the setpoint, generate a control signal to cause an actuator to assert more force on a valve seal of the surgical cassette to decrease the flow rate; and
if the flow rate is less than the setpoint, generate the control signal to cause the actuator to assert less force on the valve seal of the surgical cassette to increase the flow rate.

17. The computer program product of claim 16, wherein the set of computer instructions comprise instructions executable to:
receive an indication of a level of fluid in the surgical cassette; and
determine the flow rate based on a change in the level over time.

18. The computer program product of claim 12, wherein the set of computer instructions comprise instructions executable to generate the control signal to cause the actuator to assert sufficient force on the valve seal to stop flow at the occurrence of a predefined event.

19. A surgical system comprising:
a surgical cassette comprising:
a valve seat, wherein the valve seat is configured to provide a tapered opening to a first flow passage leading to a valve chamber;
a valve seal movable between a fully opened position and fully closed position, wherein the valve seat and valve seal define a valve chamber and wherein the surgical cassette comprises the first flow passage leading to the valve chamber and a second flow passage leading to a fluid chamber of the surgical cassette;
a surgical console comprising:
a cassette receiver to receive the surgical cassette;
a sensor system;
an actuator positioned to assert a force on the valve seal when the surgical cassette is inserted in the cassette receiver; and
a controller coupled to the sensor system and the actuator, the controller configured to:
to receive an input from the sensor system;
generate a control signal to the actuator to open or close the valve seal to control a flow rate of fluid to the fluid chamber.

20. The surgical system of claim 19, wherein the valve seal is tapered.
